(19) United States
(12) Patent Application Publication
(10) Pub. No.: US 2026/0096921 A1
(43) Pub. Date: Apr. 9, 2026

(54) BATTERY POWERED HEATED EYE MASK

(71) Applicant: BLINKJOY INC., Encinitas, CA (US)

(72) Inventor: Justin Mendelson, Encinitas, CA (US)

(21) Appl. No.: 19/351,129

(22) Filed: Oct. 6, 2025

Related U.S. Application Data
(60) Provisional application No. 63/704,481, filed on Oct. 7, 2024.

(51) Int. Cl. A61F 7/08; A61F 7/00

(52) U.S. Cl. CPC A61F 7/08; A61F 7/007; A61F 2007/0004; A61F 2007/0055; A61F 2007/0093

(57) ABSTRACT

A heated eye mask apparatus for therapeutic treatment of meibomian gland dysfunction, blepharitis, dry eye disease, and related ocular conditions. The apparatus comprises a body portion with adjustable straps and a pair of detachable eye covers releasably secured to the body portion via fastening mechanisms enabling positional adjustment. Each eye cover includes at least one heating element with multiple independently controllable heating zones, allowing targeted thermal therapy to specific periocular anatomical regions. The heating elements are powered by rechargeable batteries integrated within the body portion, providing cordless operation. Flexible tethers connect the eye covers to the body portion, providing mechanical retention and electrical connectivity. A control interface comprising user-actuatable buttons enables independent activation of heating zones. Alternative embodiments may include a steam generation system providing combined heat and moisture therapy. The adjustable configuration accommodates varying facial anatomies and enables customized treatment based on individual therapeutic requirements.

### CLAIM OF PRIORITY

The present Application for Patent claims priority to U.S. Provisional Application No. 63/704,481 entitled “Battery Powered Eye Mask”, filed Oct. 7, 2024, which is hereby expressly incorporated by reference.

### FIELD

The present disclosure relates to heated eye masks for providing therapeutic heat treatment to the periocular region, and more particularly to devices and methods for treating meibomian gland dysfunction, blepharitis, chalazia, hordeola (styes), dry eye disease, and related ocular surface disorders.

### BACKGROUND

Heated eye masks are known devices for applying thermal therapy to the periocular region. The “periocular region” refers to the area of the face surrounding the eyes, encompassing the eyebrows, eyelashes, eyelids, and the skin around them.

Conventional heated eye masks typically operate by delivering warmth to the eyelids and surrounding tissues to address various ocular conditions. Heat application to the eyelid margin has been clinically demonstrated to liquefy meibum within the meibomian glands, thereby improving tear film lipid layer quality and reducing symptoms associated with meibomian gland dysfunction and evaporative dry eye disease. Thermal therapy has also been utilized to increase localized blood circulation, reduce periorbital edema, and treat inflammatory eyelid conditions including blepharitis, chalazia, and hordeola.

Existing heated eye masks suffer from several limitations. Current commercially available devices lack portable, battery-powered heating systems with user-adjustable coverage components. Known heated eye masks provide uniform heating across the entire mask surface without differentiated temperature control for distinct anatomical regions. This uniform heating approach fails to account for the different thermal sensitivity and therapeutic requirements of the eyelid margin versus the orbital rim and surrounding periocular structures. Furthermore, conventional masks employ fixed-geometry designs that cannot be adjusted to accommodate variations in facial anatomy and interpupillary distance among different users, resulting in suboptimal thermal contact and therapeutic efficacy.

Accordingly, there is a need for a heated eye mask comprising a portable power source, adjustable eye covers configured to accommodate varying facial geometries, and a plurality of independently controllable heating zones for differential thermal therapy across distinct periocular regions.

### SUMMARY

The following presents a simplified summary of one or more implementations in order to provide a basic understanding of some implementations. This summary is not an extensive overview of all contemplated implementations and is intended to neither identify key or critical elements of all implementations nor delineate the scope of any or all implementations. Its sole purpose is to present some concepts of one or more implementations in a simplified form as a prelude to the more detailed description that is presented later.

In one or more implementations, a heated eye mask apparatus is provided for therapeutic treatment of meibomian gland dysfunction, blepharitis, dry eye disease, chalazia, hordeola, and related ocular surface disorders. The apparatus comprises a body portion configured to be worn over a user's eyes and having an interior surface and an exterior surface, at least one eye cover detachably secured to the interior surface via a repositionable fastening mechanism, at least one heating element disposed within the eye cover and comprising a plurality of independently controllable heating zones, at least one rechargeable power source secured to the body portion and electrically connected to the heating element, and a control interface disposed on the body portion and operatively coupled to the heating element for enabling independent activation and deactivation of different heating zones.

The detachable fastening mechanism permits lateral and vertical repositioning of each eye cover to accommodate variations in facial anatomy and interpupillary distance among different users, thereby optimizing thermal contact between the heating elements and target periocular anatomical structures. The plurality of independently controllable heating zones enables user-selectable differential temperature application, wherein an inner heating zone can be positioned to overlay the eyelid margin and meibomian gland region, while an outer heating zone can be positioned to overlay the orbital rim and lateral periocular tissues. The control interface comprises user-actuatable control elements enabling selective activation of specific heating zones independently or in combination, providing therapeutic flexibility based on individual symptom location and treatment requirements.

In various embodiments, the apparatus further comprises flexible tethers connecting each eye cover to the body portion, wherein the tethers provide both electrical connectivity for power delivery and mechanical retention. Alternative embodiments may include dual heating element configurations positioned at different vertical distances from the user's skin, or a steam generation system comprising a water reservoir and vents for providing combined heat and moisture therapy.

### BRIEF DESCRIPTION OF THE DRAWINGS

In the following description, specific details are given to provide a thorough understanding of the embodiments. However, it will be understood by one of ordinary skill in the art that the embodiments may be practiced without these specific details.

FIG. 1 is a front view of an eye mask according to one embodiment of the present disclosure.

FIG. 2 is a rear view of the eye mask of FIG. 1.

FIG. 3 is a front view of the eye mask with a pair of eye covers secured thereto according to one embodiment of the present disclosure.

FIG. 4 is a rear view of the eye mask of FIG. 3.

FIG. 5 is a front perspective view of the eye mask of FIGS. 3-4.

FIG. 6 is a rear perspective view of the eye mask of FIGS. 3-4.

FIG. 7 is a side exploded view illustrating the eye cover assembly and associated power system components according to one embodiment of the present disclosure.

FIG. 8 is a detailed close-up view of the eye cover assembly of FIG. 7.

FIG. 9 is an exploded view illustrating the layered construction of an eye cover assembly according to one embodiment of the present disclosure.

FIG. 10 is a side view of the assembled eye mask of FIG. 4 showing the spatial relationship between the eye covers and body portion.

FIG. 11 is a view illustrating the attachment of the eye covers to the body portion via detachable fastening mechanisms according to one embodiment of the present disclosure.

FIG. 12 is a schematic electrical diagram illustrating the electrical connectivity and control architecture of the heating system according to one embodiment of the present disclosure.

FIG. 13 is an exploded view illustrating an alternative embodiment of the eye cover assembly employing a dual heating element configuration.

FIG. 14 is a schematic view of an alternative embodiment of the eye mask including a steam generation system, illustrating the fill port, water reservoir, steam diffusion components, and vents for controlled moisture release.

### DETAILED DESCRIPTION

The features, nature, and advantages of the present aspects may become more apparent from the detailed description set forth below when taken in conjunction with the drawings in which like reference characters identify correspondingly throughout.

### Overview

The present disclosure provides a heated eye mask for delivering thermal therapy to treat meibomian gland dysfunction, blepharitis, chalazia, hordeola, and dry eye disease. The eye mask comprises a battery-powered heating system, adjustable eye covers configured to accommodate varying facial anatomies, and at least two independently controllable heating zones enabling user-selectable differential temperature application across distinct periocular regions. The battery-powered configuration eliminates tethering cables and enables multiple treatment sessions per charge cycle. The adjustable eyepieces and multi-zone heating architecture permit precise localization of thermal therapy to specific anatomical structures requiring treatment.

The eye mask further comprises a user-actuatable control element operatively coupled to the heating system for selectively activating and deactivating thermal output. The heating system is configured to deliver therapeutic heat to the periocular region for treatment of dry eye disease, meibomian gland dysfunction, blepharitis, and related ocular conditions. The body portion of the eye mask is fabricated from biocompatible, dermatologically suitable materials configured to maintain conformal contact with periocular skin without causing irritation or discomfort during extended wear. A rechargeable battery assembly is integrated within the mask housing, providing electrical power to the heating elements and enabling cordless, portable operation.

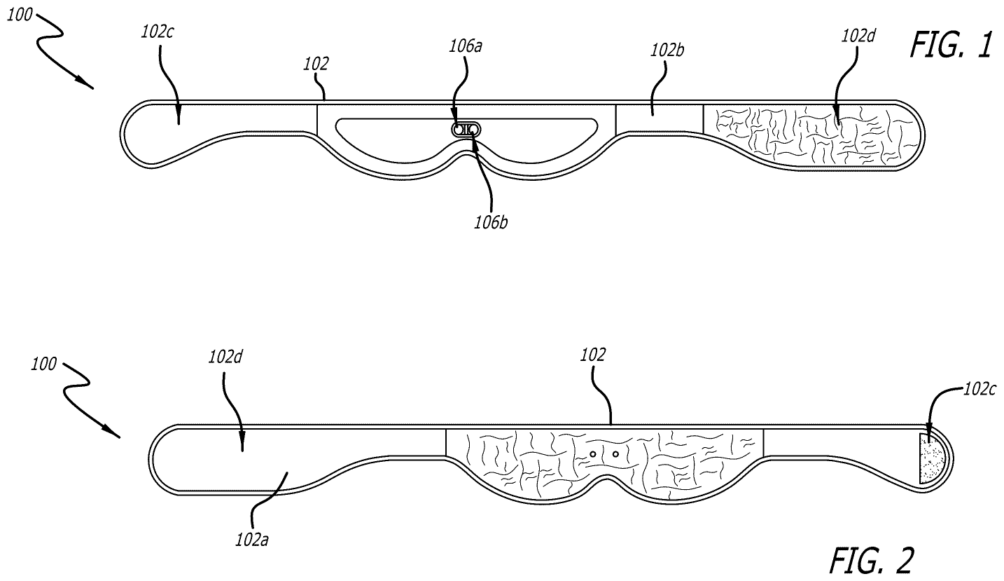

FIGS. 1-2 illustrate front and rear perspective views, respectively, of an eye mask 100 in accordance with one embodiment of the present disclosure. The eye mask 100 comprises a body portion 102 defining a generally contoured surface configured to interface with the periocular region of a user's face. The body portion 102 includes an interior surface 102a oriented toward the user during use and an opposing exterior surface 102b oriented away from the user. The interior surface 102a is configured to contact the user's facial skin in the periocular region, including areas overlying the eyelids, orbital rim, and surrounding facial structures.

The body portion 102 further comprises a first lateral strap 102c extending from a first side of the body portion 102 and a second lateral strap 102d extending from an opposing second side of the body portion 102. The first lateral strap 102c and second lateral strap 102d are configured to encircle the user's head and secure the eye mask 100 in position during use. The distal ends of the first and second lateral straps 102c, 102d are configured for releasable attachment to one another via a fastening mechanism disposed on at least one of the straps.

In various embodiments, the fastening mechanism may comprise hook-and-loop fasteners (e.g., VELCRO®), magnetic closure elements, snap fasteners, buckle assemblies, adjustable slide mechanisms, elastic bands, or combinations thereof. The use of adjustable fastening mechanisms permits the effective circumferential length of the strap assembly to be varied, thereby accommodating different head sizes and enabling the user to control the compressive force applied by the mask against the facial surface.

According to one embodiment, the body portion 102 is fabricated from a flexible, conformable material exhibiting sufficient malleability to permit manual reshaping by the user. This malleability enables the body portion 102 to be deformed to conform to individual variations in facial anatomy, including differences in nasal bridge prominence, orbital depth, cheekbone structure, and interpupillary distance. Suitable materials for the body portion 102 include, but are not limited to, thermoplastic elastomers, silicone rubber, foam materials (such as memory foam, polyurethane foam, or EVA foam), textile composites, neoprene, or multi-layer laminates combining rigid and flexible components.

In certain embodiments, the body portion 102 may incorporate a semi-rigid or selectively rigid framework embedded within or layered with flexible padding material. For example, the body portion 102 may include a malleable wire frame, a thermoplastic insert, or shaped memory metal elements that retain deformation when bent, thereby maintaining a user-customized contour. The interior surface 102a may be lined with a soft, hypoallergenic fabric layer, such as cotton, microfiber, polyester fleece, or silk, to enhance user comfort and prevent skin irritation during extended wear periods.

The exterior surface 102b may be formed from a moisture-resistant or moisture-wicking material to facilitate cleaning and prevent accumulation of perspiration or environmental contaminants. In some embodiments, the exterior surface 102b comprises a washable textile material or a wipeable polymeric surface to enable sanitation between uses.

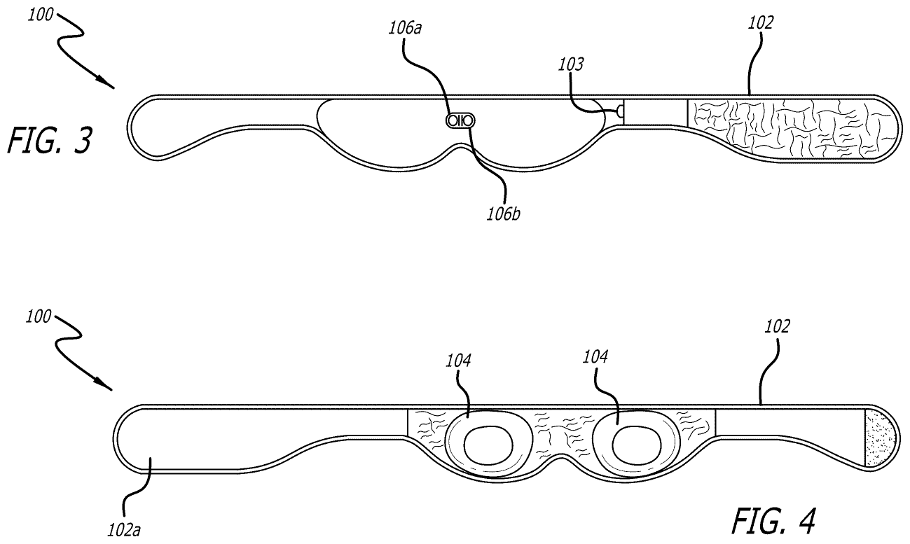

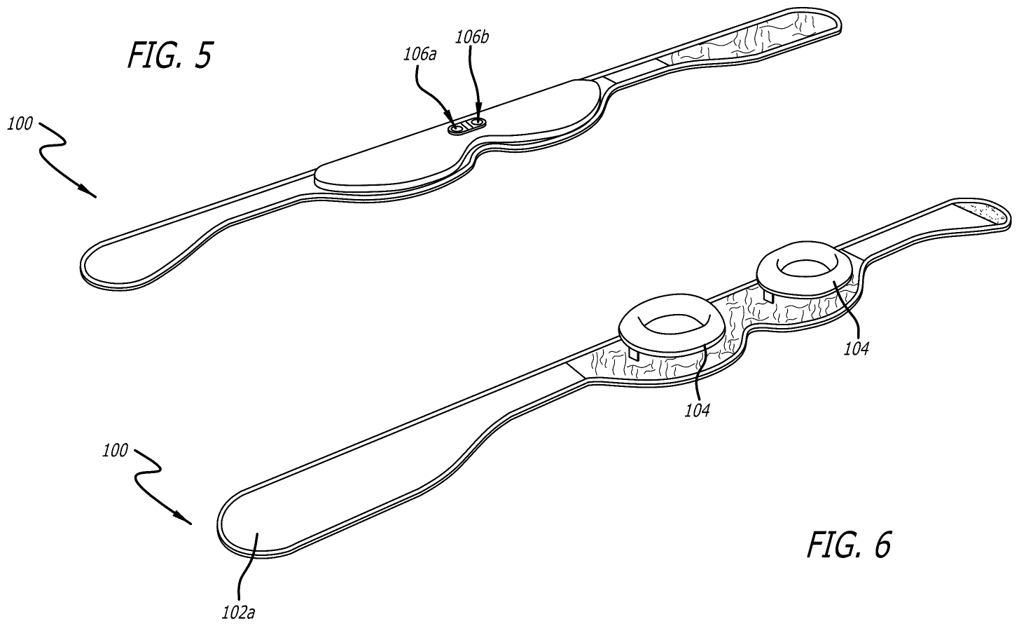

FIGS. 3-4 illustrate front and rear views, respectively, of the eye mask 100 with a pair of eye covers 104 secured to the body portion 102. FIGS. 5-6 illustrate front and rear perspective views of the assembled eye mask 100 of FIGS. 3-4. Each eye cover 104 is releasably attached to the interior surface 102a of the body portion 102 via a detachable fastening mechanism, thereby permitting removal, repositioning, and reattachment of the eye covers 104 relative to the body portion 102.

The detachable fastening mechanism enables the user to adjust the position of each eye cover 104 independently in both lateral (medial-lateral) and vertical (superior-inferior) directions relative to the body portion 102. This multi-directional adjustability permits precise alignment of the eye covers 104 with the user's individual eye positions, accounting for variations in interpupillary distance, orbital spacing, facial width, and vertical eye positioning. By enabling positional customization, the detachable configuration allows the user to optimize thermal contact between the heating elements 108 (described below) and target anatomical structures, including the eyelid margins, meibomian gland orifices, and periocular tissues.

In various embodiments, the detachable fastening mechanism may comprise hook-and-loop fasteners (e.g., VELCRO®), wherein a first fastener component is affixed to the interior surface 102a of the body portion 102 and a complementary second fastener component is affixed to the exterior surface of each eye cover 104. Alternative fastening mechanisms include, but are not limited to: magnetic attachment elements embedded in both the body portion 102 and eye covers 104; snap fastener arrays; button-and-buttonhole configurations; slide-track mechanisms; adhesive strips with repositionable adhesive; or elastic retention bands. The fastening mechanism is configured to provide sufficient retention force to maintain the eye covers 104 in position during use while permitting manual detachment and repositioning without tools.

Each eye cover 104 defines a curved or contoured aperture configured to accommodate the user's orbital anatomy. The aperture is shaped to provide clearance around the orbital rim—the bony structure circumscribing each eye socket—thereby reducing pressure points and enhancing user comfort during extended wear. In certain embodiments, the aperture comprises an oval, elliptical, or kidney-shaped opening dimensioned to surround the eye while maintaining a peripheral margin overlying the eyelids and immediately adjacent periocular tissues. The perimeter of the aperture may include a cushioned or padded edge to further enhance comfort and prevent pressure-related discomfort.

The eye mask 100 further comprises a control interface including at least one user-actuatable input element for controlling operation of the heating system. In the illustrated embodiment, the control interface comprises a first control button 106a and a second control button 106b disposed on the exterior surface 102b of the body portion 102. The positioning of the control buttons 106a, 106b on the front-facing exterior surface facilitates intuitive access by the user during operation without requiring removal of the eye mask 100.

Each control button 106a, 106b is operatively coupled to electrical circuitry configured to selectively activate and deactivate one or more heating elements 108 embedded within or attached to the eye covers 104. In one embodiment, each eye cover 104 contains at least one heating element 108, wherein the first control button 106a controls the heating element(s) in the first eye cover 104a and the second control button 106b independently controls the heating element(s) in the second eye cover 104b. This independent control architecture enables the user to apply heat therapy to one eye selectively or to both eyes simultaneously, and permits differential treatment of each eye based on individual therapeutic requirements.

The heating elements 108 may comprise flexible resistive heating elements fabricated from electrically conductive materials. Suitable heating element technologies include, but are not limited to: graphene-based heating films; carbon fiber heating pads; etched foil resistive heaters; printed conductive ink circuits; thin-film resistive heating elements; conductive polymer heating layers; wire-based heating coils (such as nichrome wire or copper wire); positive temperature coefficient (PTC) heating elements; or hybrid combinations thereof. In preferred embodiments, the heating elements 108 comprise graphene heating films due to their uniform heat distribution characteristics, rapid heating response, flexibility, and biocompatibility.

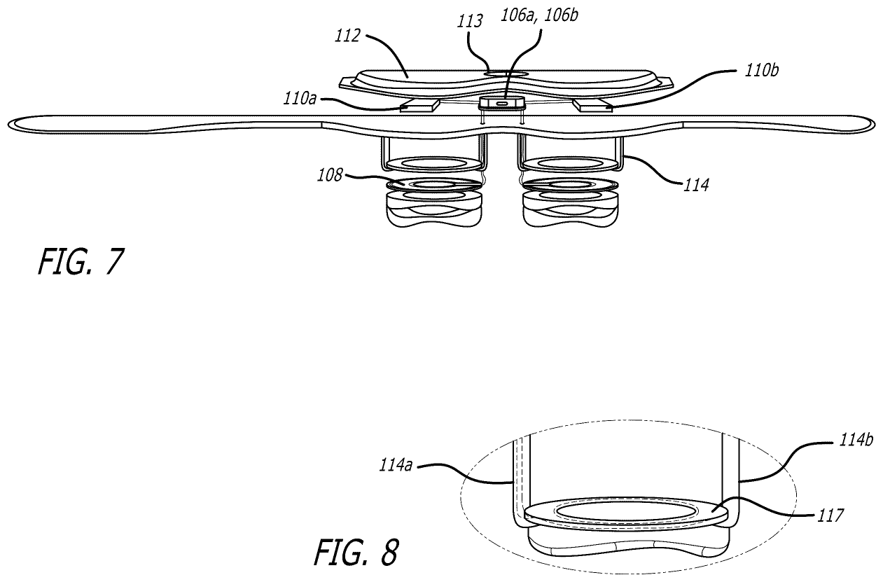

Each heating element 108 is electrically connected to a power supply circuit via flexible electrical conductors, such as insulated wires, conductive traces on flexible printed circuit boards, or conductive textile pathways. The electrical connections extend from the heating elements 108 through the eye covers 104 and body portion 102 to a battery module 110 (described below with reference to FIGS. 7-8) and control circuitry. In embodiments employing detachable eye covers 104, the electrical connections may incorporate quick-disconnect connectors, spring-loaded contact pins, conductive snap fasteners, or magnetic electrical contacts to permit electrical continuity when the eye covers 104 are attached and automatic disconnection when removed.

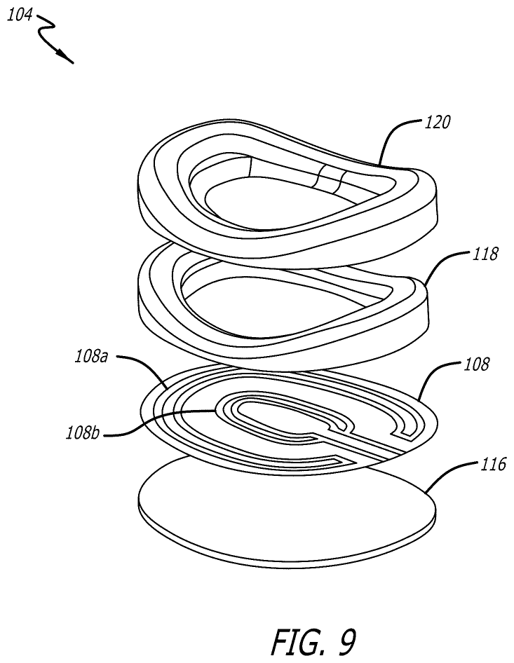

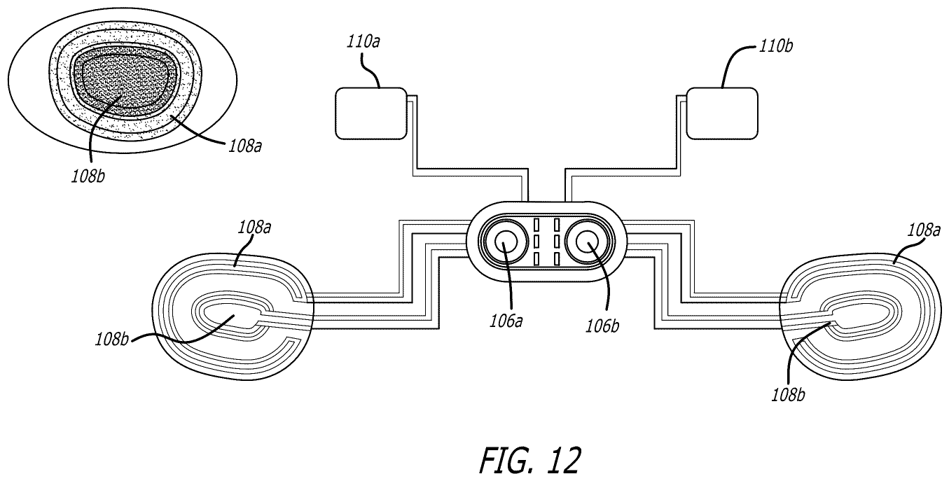

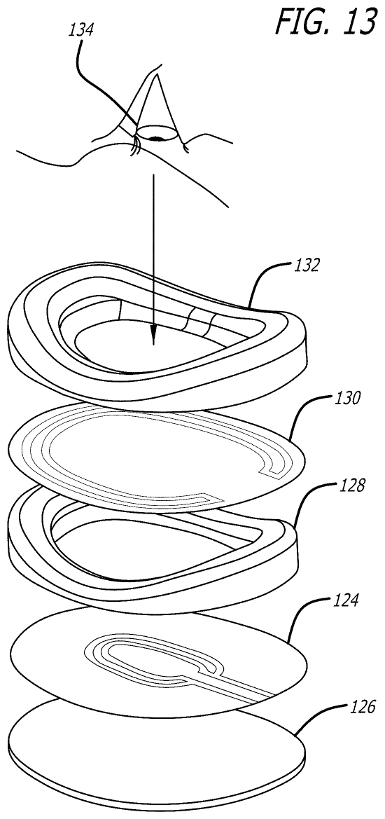

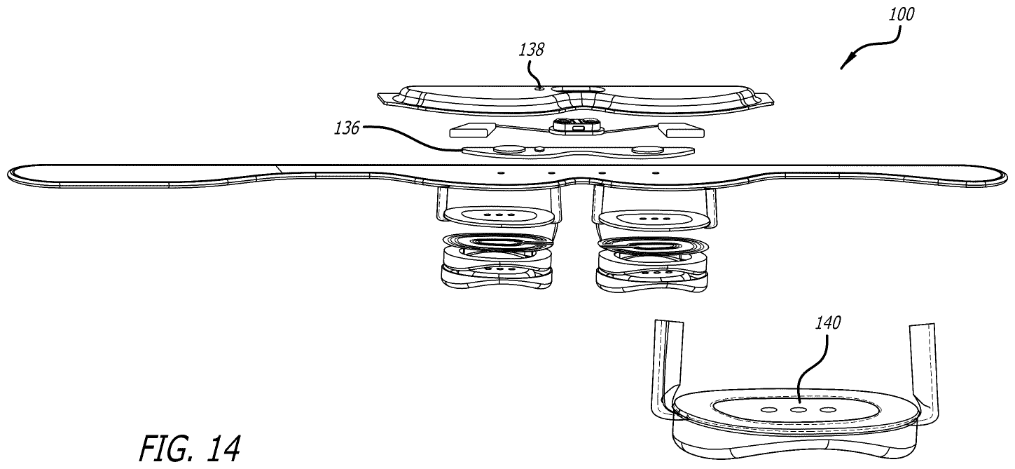

In an alternative embodiment illustrated in FIGS. 9 and 12, each eye cover 104 comprises a multi-zone heating configuration including a plurality of independently controllable heating zones. For example, each eye cover 104 may include a first heating zone 108a positioned to overlay the eyelid margin and meibomian gland region, and a second heating zone 108b positioned to overlay the orbital rim or lateral periocular tissue. The multi-zone configuration enables differential temperature control across anatomically distinct regions, thereby permitting targeted thermal therapy.

In this multi-zone embodiment, the control buttons 106a, 106b may be configured to control corresponding heating zones across both eye covers 104 rather than controlling each eye cover independently. Specifically, actuation of the first control button 106a may simultaneously activate the first heating zones 108a in both the first eye cover 104a and the second eye cover 104b, while actuation of the second control button 106b simultaneously activates the second heating zones 108b in both eye covers. This zone-based control scheme enables the user to selectively heat the inner (medial) eyelid regions, the outer (lateral) periocular regions, or both regions concurrently in a bilaterally symmetric manner.

In certain embodiments, the control buttons 106a, 106b may comprise momentary switches, toggle switches, capacitive touch sensors, or multi-position switches. The control circuitry may be configured to provide multiple operational modes accessible through the control buttons, such as: on/off control; variable intensity settings (e.g., low, medium, high heat); timed operation modes with automatic shutoff after a predetermined duration (e.g., 10, 15, or 20 minutes); pulsed heating modes with alternating on/off cycles; or temperature-regulated modes maintaining a target temperature based on feedback from temperature sensors.

In embodiments incorporating temperature sensing and regulation, each heating zone may include a temperature sensor, such as a thermistor, thermocouple, or resistance temperature detector (RTD), positioned proximate to or integrated within the heating element 108. The temperature sensor provides feedback to a control circuit, which modulates electrical power delivered to the heating element 108 to maintain a desired target temperature or prevent overheating. Typical therapeutic temperature ranges for eyelid thermal therapy are between approximately 40° C. and 45° C., which are sufficient to melt meibum while remaining safe for eyelid tissues. In some implementations, the system may be configurable to operate across a broader range to accommodate patient-specific needs.

The eye covers 104 may be fabricated from thermally conductive materials to facilitate heat transfer from the heating elements 108 to the user's skin, or may incorporate thermally insulating layers on the exterior surface to direct heat preferentially toward the interior surface contacting the user. In various embodiments, each eye cover 104 comprises a multi-layer construction including: a thermally conductive inner layer (e.g., silicone, graphene-infused polymer); the heating element 108 embedded within or laminated to an intermediate layer; and an outer cover layer providing structural support and attachment surface for the fastening mechanism. The layers may be bonded via adhesive, thermal welding, ultrasonic welding, or mechanical stitching.

In certain embodiments, the eye covers 104 may be disposable or replaceable components supplied as consumable accessories, while in other embodiments, the eye covers 104 comprise durable, cleanable components intended for repeated use. Disposable eye covers 104 may include integrated heating elements and electrical connectors designed for limited-use applications, while reusable eye covers 104 may incorporate more robust heating elements and detachable, washable fabric liners.

Referring to FIG. 7, a side exploded view of the eye cover assembly 104 and associated power system components is illustrated. FIG. 8 provides a detailed close-up view showing the spatial relationship between the components of the eye cover 104. As shown in these figures, the eye mask 100 comprises a dual power source configuration including a first power source 110a electrically coupled to the first control button 106a and a second power source 110b electrically coupled to the second control button 106b. Each power source 110a, 110b is independently operable to provide electrical power to its respective heating element(s) 108, thereby enabling independent control of heating operations for different heating zones or eye covers as previously described.

In various embodiments, each power source 110a, 110b comprises a rechargeable electrochemical battery. Suitable battery technologies include, but are not limited to: lithium-ion batteries; lithium-polymer (LiPo) batteries; nickel-metal hydride (NiMH) batteries; or other rechargeable battery chemistries exhibiting suitable energy density, charge retention, and cycle life characteristics. In preferred embodiments, the power sources 110a, 110b comprise lithium-polymer batteries due to their compact form factor, light weight, flexible packaging options, and high energy density.

Each power source 110a, 110b comprises a rechargeable electrochemical battery having sufficient capacity to power the heating elements for multiple therapeutic sessions before requiring recharging.

The power sources 110a, 110b and control buttons 106a, 106b are secured to the exterior surface 102b of the body portion 102 by a retention assembly. In the illustrated embodiment, the retention assembly comprises a molded foam member 112 overlying the power sources 110a, 110b and control buttons 106a, 106b. The foam member 112 is preferably fabricated from ethylene-vinyl acetate (EVA) foam, a closed-cell foam material exhibiting desirable properties including flexibility, resilience, chemical resistance, cushioning, and ease of molding. The EVA foam member 112 serves multiple functions: (i) mechanically retaining the power sources 110a, 110b and control buttons 106a, 106b against the exterior surface 102b; (ii) providing cushioning and shock absorption to protect the power sources from impact; (iii) distributing pressure evenly across the components; and (iv) providing a finished aesthetic appearance to the exterior of the device.

Alternative materials for the foam member 112 include polyurethane foam, polyethylene foam, neoprene, silicone rubber, thermoplastic elastomer, or composite materials combining foam layers with fabric or polymeric outer layers. The foam member 112 has a thickness selected to provide adequate cushioning and component retention while maintaining a low-profile configuration suitable for comfortable wear.

The foam member 112 defines one or more apertures or openings 113 (see FIG. 7) aligned with the control buttons 106a, 106b. Each aperture 113 is dimensioned to permit the actuatable portion of the respective control button 106a, 106b to protrude through the foam member 112, thereby providing direct user access to the buttons. The aperture(s) 113 may be precisely molded during formation of the foam member 112, or may be cut, punched, or otherwise formed after molding. The perimeter of each aperture 113 may closely conform to the shape of the corresponding control button to prevent ingress of moisture or contaminants while permitting button actuation.

As shown in FIG. 8, the eye cover 104 includes a hook-and-loop fastener element 117 disposed on the exterior surface of the eye cover. The hook-and-loop fastener element 117 comprises one component of the detachable fastening mechanism previously described, configured to mate with a complementary hook-and-loop fastener component affixed to the interior surface 102a of the body portion 102. The hook-and-loop fastener element 117 enables the user to easily detach and reattach the eye cover 104 from the body portion 102 for repositioning, cleaning, or replacement. The fastener element 117 may comprise either the hook component or the loop component of the hook-and-loop fastener pair, with the complementary component disposed on the body portion 102. The hook-and-loop fastener configuration provides sufficient holding force to maintain the eye cover 104 securely in position during use while permitting manual detachment without tools or

In certain embodiments, the control buttons 106a, 106b may include tactile features such as raised surfaces, textured patterns, or distinct shapes (e.g., circular vs. rectangular) to enable the user to distinguish between buttons by touch without visual reference, thereby facilitating operation while the mask is being worn.

According to one embodiment, the foam member 112 is releasably attached to the exterior surface 102b of the body portion 102 via a detachable fastening mechanism. This detachable configuration permits removal of the foam member 112 to access the underlying power sources 110a, 110b for replacement, maintenance, or recharging operations. Suitable fastening mechanisms include: hook-and-loop fasteners; snap fasteners; magnetic attachment elements; flexible clips or tabs molded into the foam member 112 that engage corresponding retention features on the body portion 102; adhesive strips with repositionable adhesive; elastic retention bands; or drawstring closures.

In embodiments where the power sources 110a, 110b are removable for external charging, the foam member 112 and/or body portion 102 includes an access opening or removable panel to facilitate battery extraction and insertion. Alternatively, in embodiments where the power sources 110a, 110b are charged in situ, the device includes a charging interface or port 103 (See FIG. 3) accessible without removing the foam member 112. The charging interface or port 103 may be a USB Type-C port, micro-USB port, or proprietary charging connector, disposed on the body portion 102 or foam member 112 and electrically connected to the power sources 110a, 110b via charging circuitry. The charging circuitry may include battery management components such as charge controllers, overcharge protection circuits, thermal monitoring, and cell balancing circuitry for multi-cell battery configurations.

In alternative embodiments, the charging interface or port 103 comprises wireless charging functionality, wherein the eye mask includes an inductive charging coil positioned to receive electromagnetic energy from an external wireless charging pad. The inductive charging coil is electrically coupled to the power sources 110a, 110b through appropriate rectification and regulation circuitry to convert received AC power to DC charging current.

The eye covers 104 are mechanically and electrically connected to the body portion 102 via a tether assembly. Specifically, a first tether 114a couples the first eye cover 104a to the body portion 102, and a second tether 114b couples the second eye cover 104b to the body portion 102. Each tether 114a, 114b serves a dual function: (i) mechanically connecting the eye cover to the body portion 102 to prevent loss or separation during use; and (ii) providing an electrical pathway for power and control signals between the power sources 110a, 110b, control circuitry, and heating elements 108 within the respective eye covers 104.

Each tether 114a, 114b comprises a flexible elongate member having a proximal end attached to the body portion 102 and a distal end attached to the corresponding eye cover 104. The tether may comprise a flexible sleeve, tube, or sheath enclosing one or more insulated electrical conductors (wires) extending between the body portion 102 and eye cover 104. The electrical conductors provide electrical power from the power sources 110a, 110b to the heating elements 108 and may also carry control signals or sensor data if the eye covers include temperature sensors or other sensing elements.

In various embodiments, each tether 114a, 114b has a length selected to provide sufficient slack to permit the adjustable positioning of the eye covers 104 described previously while maintaining organized routing of the electrical connections. The tether material may comprise flexible polymers such as silicone, thermoplastic polyurethane (TPU), PVC, braided textile sleeves, or fabric-covered wiring.

The attachment points between the tethers 114a, 114b and the body portion 102 may comprise strain relief features to prevent mechanical stress on the electrical connections during flexing, pulling, or repositioning of the eye covers 104. Strain relief may be provided by molded polymer boots, spiral cable wraps, flexible grommet inserts, or reinforced attachment zones with increased material thickness.

In embodiments employing detachable eye covers 104 with repositionable attachment as previously described, the tethers 114a, 114b are sufficiently flexible and of adequate length to permit movement of the eye covers across the range of adjustment positions on the interior surface 102a without imposing excessive tension or causing disconnection. The tether routing may be configured such that the electrical conductors do not interfere with the fastening mechanism or create uncomfortable pressure points against the user's face.

The electrical connections at the distal ends of the tethers 114a, 114b may terminate in connectors that mate with corresponding receptacles on the eye covers 104, or the conductors may be permanently bonded or soldered to electrical terminals on the heating elements 108. In embodiments with permanent electrical connections, the eye covers 104 and tethers form an integrated subassembly that is not user-separable but may be detached from the body portion 102 as a unit (e.g., for washing or replacement).

The power distribution architecture may be configured such that the first power source 110a supplies power to the first eye cover 104a via the first tether 114a, and the second power source 110b independently supplies power to the second eye cover 104b via the second tether 114b. This dual independent power source configuration provides several advantages: (i) balanced weight distribution with power sources positioned symmetrically on the body portion 102; (ii) electrical isolation between eye covers, reducing risk of total system failure if one power source fails; (iii) independent control and power management for each eye; and (iv) redundancy enabling continued single-eye operation if one power source is depleted or malfunctioning.

Alternatively, in certain embodiments, a single centrally-positioned power source may supply power to both eye covers 104 via respective tethers, or the two power sources 110a, 110b may be electrically connected in parallel to provide combined power capacity with redundancy.

The device may further include visual indicators, such as light-emitting diodes (LEDs), disposed on the exterior surface 102b to communicate operational status to the user. For example, separate LEDs may indicate: power on/off status; heating element activation status; battery charge level (e.g., multi-color LEDs showing green for full charge, yellow for partial charge, red for low charge); charging status; or fault conditions. The LEDs may be positioned adjacent to the control buttons 106a, 106b, integrated into the buttons themselves, or positioned elsewhere on the exterior surface 102b where visible to the user.

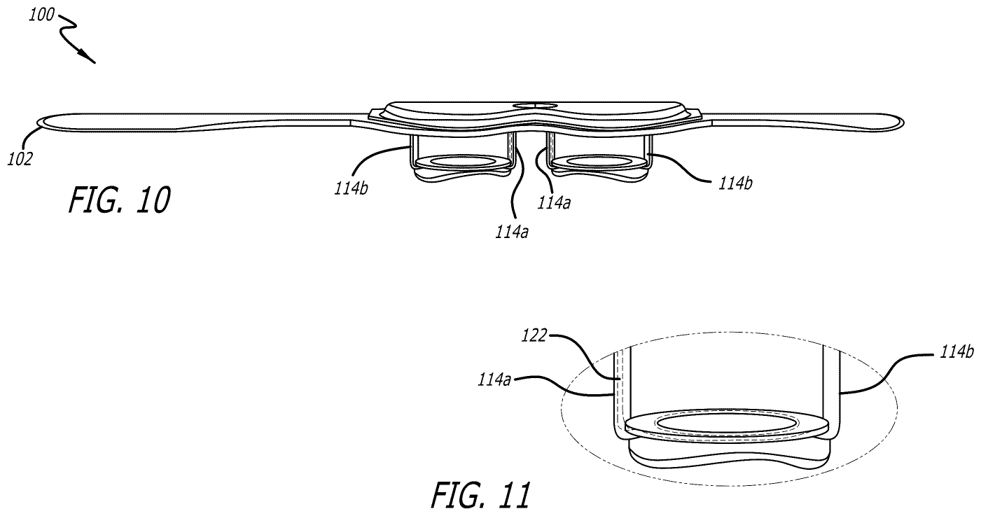

Referring to FIG. 9, an exploded view of the eye cover assembly 104 is illustrated, showing the layered construction of the eye cover components. FIG. 10 illustrates a side view of the assembled eye mask 100 of FIG. 4, showing the spatial relationship between the eye covers 104 and the body portion 102. As shown in FIG. 9, each eye cover 104 comprises a multi-layer construction including a heating element 108, a cushioning pad 116, an annular foam member 118, and a contoured foam cup 120, arranged in a stacked configuration.

The heating element 108 is disposed on or secured to the cushioning pad 116, which forms the base layer of the eye cover assembly. The cushioning pad 116 provides structural support for the heating element 108 and facilitates thermal transfer from the heating element to the user's periocular tissues during use. In various embodiments, the cushioning pad 116 may comprise thermally conductive foam materials such as open-cell or closed-cell polyurethane foam, EVA foam, silicone foam, or composite materials including thermally conductive fillers (e.g., graphite particles, carbon nanotubes, or metallic particles) to enhance heat distribution.

The heating element 108 comprises a multi-zone heating configuration including at least two independently controllable heating zones. In the illustrated embodiment, the heating element 108 includes an outer heating zone 108a and an inner heating zone 108b, each zone being electrically independently addressable. The outer heating zone 108a is positioned to overlay anatomical structures including the lateral periocular region, orbital rim, and lateral eyelid margin when the eye cover 104 is properly positioned on the user's face. The inner heating zone 108b is positioned to overlay the central eyelid region, including the area directly overlying the meibomian gland orifices at the eyelid margin.

Each heating zone 108a, 108b may comprise separate heating elements electrically isolated from one another, or may comprise distinct regions of a continuous heating element wherein different regions are separately addressable through electrical switching circuitry. For example, the heating element 108 may comprise a graphene heating film with separate electrical contact pads or bus bars defining the outer zone 108a and inner zone 108b, wherein each zone receives electrical power through dedicated conductors routed through the tether 114.

The independent control of the outer and inner heating zones 108a, 108b enables the user to selectively activate thermal therapy to specific anatomical regions based on therapeutic needs. For example, a user experiencing meibomian gland dysfunction primarily affecting the central eyelid margin may activate only the inner heating zone 108b, thereby concentrating thermal energy at the treatment site while avoiding unnecessary heating of surrounding tissues. Conversely, a user experiencing generalized eyelid inflammation or seeking broader periocular warming may activate both zones simultaneously. The user may also activate only the outer zone 108a for treatment focused on the lateral periocular region or orbital area.

In alternative embodiments, the heating element 108 may comprise three or more independently controllable heating zones. For example, the heating element may include a medial zone positioned toward the nasal side of the eye, a central zone overlying the eyelid, and a lateral zone positioned toward the temporal side of the eye. Additional zone configurations may include superior and inferior zones, concentric annular zones, or gridded zones providing fine-grained spatial control of heat delivery.

Overlying the heating element 108 is an annular foam member 118, also referred to herein as a foam donut 118 due to its toroidal or ring-like configuration. The foam donut 118 defines a central aperture that aligns with the user's eye when the eye cover 104 is positioned on the face. The foam donut 118 provides cushioning and distributes pressure evenly around the orbital rim, preventing concentrated pressure points that could cause discomfort during extended wear. The annular configuration of the foam donut 118 ensures that cushioning support is provided around the eye periphery while maintaining clearance over the eye itself.

The foam donut 118 may be fabricated from soft, resilient foam materials including memory foam, polyurethane foam, EVA foam, latex foam, or viscoelastic foam. In preferred embodiments, the foam donut 118 comprises medical-grade memory foam that conforms to the user's unique facial contours over time, thereby enhancing comfort and improving thermal contact between the heating element 108 and the user's skin. The foam donut 118 has sufficient thickness to provide adequate cushioning while maintaining a low-profile configuration.

Secured atop the foam donut 118 is a contoured foam cup 120. The foam cup 120 comprises a three-dimensional molded or shaped structure configured to conform to the anatomical contours of the periocular region, including the curvature of the orbital rim, the prominence of the eyeball, and the contours of the eyelids. The foam cup 120 defines a concave interior surface that mirrors the convex shape of the eye and surrounding orbital anatomy, thereby distributing applied pressure evenly across the contact surface when the eye mask 100 is worn.

The contoured configuration of the foam cup 120 serves multiple functions: (i) enhancing user comfort by eliminating pressure concentration points; (ii) improving thermal contact between the heating element 108 and target tissues by conforming the flexible heating element to the facial surface; (iii) providing a finished aesthetic appearance to the eye cover 104; and (iv) protecting the underlying heating element 108 and electrical connections from mechanical damage. The foam cup 120 may include a smooth interior surface that contacts the user's skin and an exterior surface that may be textured, covered with fabric, or finished with a protective coating.

In various embodiments, the foam cup 120 may be fabricated from the same or different foam materials as the foam donut 118. The foam cup 120 may comprise a unitary molded structure or may be formed from multiple layers laminated or bonded together. In certain embodiments, the foam cup 120 includes an outer shell or cover layer comprising fabric (e.g., cotton, polyester, microfiber, or moisture-wicking athletic fabrics), leather, synthetic leather, or polymeric materials (e.g., silicone, thermoplastic elastomer) for enhanced durability and cleanability.

The layers of the eye cover assembly 104 (cushioning pad 116, heating element 108, foam donut 118, and foam cup 120) may be secured together using various bonding techniques including adhesive bonding, thermal welding, ultrasonic welding, mechanical fasteners, stitching, or combinations thereof. In embodiments where the foam cup 120 includes a removable fabric cover, the cover may be detachable for washing via zipper closures, hook-and-loop fasteners, snap fasteners, or elastic retention bands.

As shown in FIGS. 10-11, each eye cover 104 is mechanically connected to the body portion 102 of the eye mask 100 via the first tether 114a and the second tether 114b. The tethers 114a, 114b extend from attachment points on the body portion 102 to corresponding attachment points on the eye cover 104, thereby maintaining a physical connection between the eye cover and the main body of the device while permitting limited movement of the eye cover for positional adjustment as previously described.

As shown in FIGS. 10-11, each eye cover 104 is mechanically connected to the body portion 102 of the eye mask 100 via the first tether 114a and the second tether 114b. The tethers 114a, 114b extend from attachment points on the body portion 102 to corresponding attachment points on the eye cover 104, thereby maintaining a physical connection between the eye cover and the main body of the device while permitting limited movement of the eye cover for positional adjustment as previously described.

According to one embodiment, the first tether 114a and second tether 114b comprise elongate flexible members having retractable or extensible properties. Each tether 114a, 114b is configured to permit controlled extension when the eye cover 104 is pulled away from the body portion 102 (e.g., during repositioning or adjustment), and to retract or return toward an unextended state when pulling force is released. This retractable functionality maintains organized cable management, prevents excessive slack in the tethers that could become tangled or caught, and provides gentle restoring force that assists in maintaining the eye cover in position.

The retractable tethers 114a, 114b may be implemented using various mechanisms including: coiled or spiral cable configurations that extend when pulled and contract when released; elastic materials or fabrics that stretch under tension and return to original length when tension is released; spring-loaded retractor mechanisms similar to those used in retractable badge holders or measuring tapes; or combinations thereof. In preferred embodiments, the tethers comprise coiled electrical cables (similar to coiled telephone cords) that provide both electrical connectivity and mechanical retractability in a single integrated component.

Each tether 114a, 114b has a maximum extension length that limits the distance the eye cover 104 can be displaced from the body portion 102. When the eye cover 104 is pulled outward to the maximum extension length permitted by the tethers, the tethers provide increasing resistance or tension that prevents further extension and signals to the user that the adjustment limit has been reached. This maximum extension limit prevents over-extension that could damage electrical connections or detach the eye cover from the body portion 102 unintentionally.

The tethers 114a, 114b may be fabricated from various materials selected for flexibility, durability, and electrical conductivity (where applicable). Suitable materials include braided fabric sleeves, silicone tubing, thermoplastic elastomer, spiral-wound polymers, or textile-covered electrical cables. In embodiments employing elastic retraction, the tether material may comprise elastomeric polymers such as spandex, natural rubber, silicone rubber, or thermoplastic elastomer exhibiting suitable elastic recovery properties.

According to one embodiment, the first tether 114a comprises an electrical tether incorporating one or more insulated electrical conductors (wires) extending internally through the length of the tether between the power source (110a or 110b) and the heating element 108 within the eye cover 104. The electrical conductors provide the electrical pathway for delivering power from the battery to the heating zones 108a, 108b, and may also carry control signals or sensor feedback signals if the eye cover includes temperature sensors or other electronic components.

The electrical conductors within the first tether 114a may comprise stranded copper wire, tinned copper wire, or other conductive materials exhibiting suitable flexibility and conductivity. The conductors are individually insulated with polymer insulation (e.g., PVC, silicone, PTFE) to prevent electrical shorts, and multiple conductors may be bundled within a common outer sheath forming the tether structure. For a dual-zone heating element configuration, the first tether 114a may contain multiple conductors, for example: a positive supply conductor for the outer zone 108a, a positive supply conductor for the inner zone 108b, and one or more common ground/return conductors.

The second tether 114b, according to one embodiment, serves primarily as a mechanical retention tether providing redundant physical connection between the eye cover 104 and body portion 102. The second tether 114b may not contain electrical conductors, instead comprising solely a flexible mechanical retention element. This dual-tether configuration provides important safety and reliability benefits: if the user inadvertently applies excessive pulling force to the eye cover 104, the second tether 114b shares the mechanical load with the first tether 114a, thereby reducing stress on the electrical connections within the first tether and preventing wire breakage or disconnection.

The second tether 114b effectively functions as a strain relief element, protecting the electrical integrity of the first tether 114a from mechanical damage due to user handling. By distributing tensile forces across two separate tethers, the likelihood of electrical connection failure is significantly reduced, enhancing device reliability and longevity.

In alternative embodiments, both the first tether 114a and second tether 114b may contain electrical conductors, providing redundant electrical pathways. For example, each tether may carry power to different heating zones, or may provide parallel redundant connections to the same heating element for enhanced reliability. In yet another embodiment, a single tether incorporating both electrical conductors and reinforced mechanical strain relief may be employed in place of the dual-tether configuration.

The attachment points where the tethers 114a, 114b connect to the body portion 102 and eye cover 104 may include strain relief features such as molded polymer boots, reinforced grommets, or anchor points with increased material thickness to prevent mechanical fatigue or failure at these high-stress locations. The electrical connections at the tether terminations may be soldered, crimped, or connected via miniature electrical connectors designed for reliable connection in flexible cable applications.

In certain embodiments, the maximum extension length of the retractable tethers 114a, 114b may be user-adjustable. For example, the tethers may include locking mechanisms that permit the user to set a desired maximum extension length based on their facial dimensions and preferred eye cover positioning, then lock the tether at that length to prevent further extension during use.

Referring to FIG. 12, a schematic electrical diagram illustrating the electrical connectivity and control architecture of the heating system is shown. The schematic depicts the electrical connections between the power sources 110a, 110b, control buttons 106a, 106b, and heating elements 108 within the eye covers 104. As illustrated, the heating elements 108 are operatively coupled to the power sources 110a, 110b through the control interface comprising the first control button 106a and second control button 106b, which function as user-actuatable switching elements to selectively enable or disable electrical current flow to specific heating zones.

In the illustrated embodiment, each heating element 108 comprises a dual-zone configuration including an outer heating zone 108a and an inner heating zone 108b. The outer heating zones 108a of both the first eye cover 104a and second eye cover 104b are electrically connected in parallel or series and coupled to the first control button 106a. Similarly, the inner heating zones 108b of both eye covers are electrically connected and coupled to the second control button 106b. This zone-based control architecture enables bilateral symmetric heating control, wherein actuation of the first control button 106a simultaneously activates or deactivates the outer zones 108a of both eye covers, and actuation of the second control button 106b simultaneously activates or deactivates the inner zones 108b of both eye covers.

Each control button 106a, 106b functions as an electrical switch element that selectively completes or interrupts the electrical circuit between the respective power source and the corresponding heating zones. When a control button is actuated to an “ON” state, the switch closes, allowing electrical current to flow from the power source through the heating element, thereby generating resistive heat. When the control button is actuated to an “OFF” state, the switch opens, interrupting current flow and terminating heat generation.

The control buttons 106a, 106b may comprise various switching technologies including: momentary push-button switches requiring continuous depression to maintain the ON state; latching push-button switches that toggle between ON and OFF states with successive actuations; slide switches; rocker switches; capacitive touch switches; or membrane switches. In preferred embodiments, the control buttons comprise latching push-button switches that maintain their state (ON or OFF) after actuation without requiring continuous user input, thereby enabling hands-free operation once the desired heating zones are activated.

According to one embodiment, the user can selectively activate the outer heating zones 108a only (by actuating the first control button 106a while leaving the second control button 106b in the OFF state), the inner heating zones 108b only (by actuating the second control button 106b while leaving the first control button 106a in the OFF state), both outer and inner heating zones simultaneously (by actuating both control buttons 106a and 106b to the ON state), or neither zone (by maintaining both control buttons in the OFF state). This four-state control capability provides therapeutic flexibility, enabling the user to customize heat delivery based on symptom location, treatment objectives, and personal comfort preferences.

In alternative embodiments, the control architecture may be configured such that each control button 106a, 106b independently controls the heating element(s) within a single eye cover rather than controlling a specific zone across both eye covers. For example, the first control button 106a may control all heating zones within the first eye cover 104a (right eye), while the second control button 106b controls all heating zones within the second eye cover 104b (left eye). This eye-based control scheme enables unilateral treatment, wherein the user can apply heat therapy to one eye independently of the other eye.

The electrical connections between the power sources 110a, 110b and the heating zones may be implemented using various circuit topologies. In one embodiment, the first power source 110a supplies electrical power to the outer heating zones 108a through the first control button 106a, while the second power source 110b independently supplies power to the inner heating zones 108b through the second control button 106b. This dual independent power source configuration provides electrical isolation between heating zones and enables continued operation of one zone if the other power source is depleted or malfunctioning.

Alternatively, a single power source may supply both heating zones through separate control switches, or the two power sources 110a, 110b may be electrically connected in parallel to provide combined power capacity distributed to the heating zones through appropriate switching and distribution circuitry.

While the illustrated embodiment depicts a dual-zone heating configuration (outer zone 108a and inner zone 108b) for each eye cover 104, the heating system architecture is adaptable to accommodate different numbers of independently controllable heating zones. In simplified embodiments, each eye cover 104 may comprise a single heating zone (one heating element per eye cover) without zone differentiation, thereby requiring only two control states (heating ON or OFF) rather than the four-state control enabled by dual-zone configurations.

In more complex embodiments, each eye cover 104 may comprise three or more independently controllable heating zones. For example, a tri-zone configuration may include: a medial zone positioned toward the nasal side of the eye; a central zone overlying the eyelid margin; and a lateral zone positioned toward the temporal side of the eye. A quad-zone configuration may further subdivide into superior and inferior zones, providing four independently addressable regions per eye cover. Multi-zone configurations with five, six, or more zones may provide increasingly fine-grained spatial control of heat delivery, approaching a pixelated or gridded heating array.

For configurations with more than two heating zones, the control interface may be expanded to include additional control buttons or switches, or may employ alternative control mechanisms such as: multi-position rotary switches or slide switches enabling selection among multiple zone combinations; touchscreen interfaces displaying a graphical representation of the eye cover with selectable zone regions; smartphone application interfaces communicating with the device via wireless protocols (Bluetooth, Wi-Fi); voice control interfaces; or sequential button presses on a single multi-function button to cycle through available zone configurations.

The electrical circuit may further include control circuitry disposed between the control buttons 106a, 106b and the heating elements 108. The control circuitry may comprise discrete electronic components, integrated circuits, or microcontroller-based systems implementing various control functions including: pulse-width modulation (PWM) for variable power output and temperature control; timed operation modes with automatic shutoff after predetermined durations (e.g., 5, 10, 15, or 20 minutes); temperature regulation based on feedback from temperature sensors; safety shutdown in response to over-temperature conditions or circuit faults; battery charge monitoring and low-battery warnings; or user-selectable intensity settings (low, medium, high heat output).

In embodiments incorporating temperature sensing and regulation, each heating zone 108a, 108b may include an associated temperature sensor positioned proximate to or integrated within the heating element. Suitable temperature sensors include thermistors (negative or positive temperature coefficient), thermocouples, resistance temperature detectors (RTDs), or semiconductor-based temperature sensors. The temperature sensor provides electrical signals indicative of the heating element temperature to a control circuit, which compares the measured temperature to a target temperature setpoint and modulates electrical power delivered to the heating element to maintain the desired temperature.

In embodiments incorporating temperature sensing and regulation, each heating zone 108a, 108b may include an associated temperature sensor positioned proximate to or integrated within the heating element. Suitable temperature sensors include thermistors (negative or positive temperature coefficient), thermocouples, resistance temperature detectors (RTDs), or semiconductor-based temperature sensors. The temperature sensor provides electrical signals indicative of the heating element temperature to a control circuit, which compares the measured temperature to a target temperature setpoint and modulates electrical power delivered to the heating element to maintain the desired temperature.

Temperature regulation may be implemented using feedback control algorithms. The target temperature setpoint may be factory-preset to a therapeutically effective temperature for eyelid thermal therapy, or may be user-adjustable through additional control interface elements enabling selection among multiple temperature settings.

The electrical circuit may include safety protection features such as: over-temperature cutoff switches or thermal fuses that permanently or temporarily disconnect power if the heating element exceeds a maximum safe temperature threshold; current-limiting resistors or active current-limiting circuitry preventing excessive current draw that could damage components or create safety hazards; short-circuit protection detecting and disconnecting power in response to electrical shorts; and polarity protection preventing damage if batteries are installed with reversed polarity.

The eye mask may further include visual or audible feedback mechanisms providing user indication of operational status. Visual indicators may comprise light-emitting diodes (LEDs) associated with each control button 106a, 106b, wherein the LED illuminates when the corresponding heating zone is activated. The LEDs may employ different colors to indicate different operational states, for example: green indicating normal heating operation; red indicating low battery; yellow indicating intermediate battery charge; or flashing patterns indicating fault conditions or completion of timed heating cycles.

Audible feedback may be provided through piezoelectric buzzers or speakers emitting tones, beeps, or voice prompts in response to button actuations, heating activation/deactivation, timer completion, low battery conditions, or fault conditions. Haptic feedback (vibration) may alternatively or additionally provide tactile indication of operational state changes.

In embodiments employing wireless connectivity, the device may include a wireless communication module (e.g., Bluetooth Low Energy transceiver) enabling communication with an external device such as a smartphone, tablet, or computer. A companion software application executing on the external device may provide enhanced control capabilities including: graphical zone selection interface; programmable heating schedules or treatment protocols; temperature monitoring and data logging; battery charge status monitoring; firmware updates; usage tracking and therapy adherence monitoring; or integration with health tracking platforms.

The electrical conductors routing power and control signals between the body portion 102 and eye covers 104 may comprise multi-conductor cables with individually insulated wires, flexible printed circuit boards (flex PCBs), or conductive textile pathways. The number of conductors required depends on the heating zone configuration and control architecture. For example, a dual-zone configuration may require four conductors per eye cover (positive and negative/ground for each of two zones), or may employ common ground architecture reducing the conductor count to three per eye cover (two positive supply lines and one shared ground/return).

Electrical connections between components may be established using various techniques including soldering, crimping, wire-wrapping, spring-loaded pogo pins, magnetic electrical contacts, or miniature electrical connectors (e.g., JST connectors, flex circuit connectors, board-to-board connectors). In embodiments employing detachable eye covers 104, the electrical connections may incorporate quick-disconnect connectors enabling separation and reconnection without tools.

The versatility of the heating zone configuration and control architecture enables the device to accommodate diverse therapeutic applications and user preferences. The ability to independently control different heating zones allows targeted thermal therapy delivery precisely where needed, while avoiding unnecessary heating of regions not requiring treatment, thereby conserving battery power and enhancing user comfort during extended wear periods.

Referring to FIG. 13, an exploded view of an alternative embodiment of the eye cover assembly 104 is illustrated. This alternative configuration employs a dual heating element architecture wherein heating elements are disposed at multiple depths within the layered structure of the eye cover, providing enhanced thermal distribution characteristics and improved therapeutic versatility compared to single-layer heating configurations.

In this embodiment, the eye cover 104 comprises a multi-layer construction including, from bottom to top: a cushioning pad 126 forming the base layer; a first heating element 124 disposed on the cushioning pad 126; an annular foam member 128 (foam donut) overlying the first heating element 124; a second heating element 130 disposed on the annular foam member 128; and a contoured foam cup 132 forming the outermost layer. This stacked arrangement positions heating elements at two different vertical distances from the user's skin, enabling differential heat delivery to tissues at varying depths.

The first heating element 124 is secured to or disposed upon the cushioning pad 126, which provides structural support and thermal interface properties. The cushioning pad 126 may comprise foam materials as previously described, including thermally conductive foams or composite materials facilitating heat transfer. The first heating element 124, being positioned closest to the user's skin when the eye cover is worn, delivers direct thermal energy to superficial periocular tissues including the eyelid skin, eyelid margin, and immediately underlying structures.

Overlying the first heating element 124 is the annular foam member 128 (foam donut), which defines a toroidal or ring-shaped configuration with a central aperture aligned with the user's eye. The foam donut 128 serves multiple functions in this dual-element embodiment: (i) providing cushioning and pressure distribution around the orbital rim; (ii) creating thermal insulation or thermal mass that modulates heat transfer characteristics; (iii) establishing spatial separation between the first and second heating elements 124, 130; and (iv) defining the aperture through which the eye projects.

The second heating element 130 is secured to or disposed upon the upper surface of the foam donut 128. The second heating element 130 is thus positioned at a greater distance from the user's skin compared to the first heating element 124, with the foam donut 128 interposed between them. This elevated position enables the second heating element 130 to provide broader, more diffuse heating to a larger area of the periocular region, including tissues overlying the orbital rim and surrounding facial structures.

The dual heating element configuration provides several therapeutic advantages. The first heating element 124 can deliver concentrated, direct heat to the eyelid margin and meibomian gland region where precise temperature elevation is therapeutically beneficial for liquefying meibum and treating meibomian gland dysfunction. Simultaneously or independently, the second heating element 130 can provide broader heating to the surrounding periocular area, promoting increased blood circulation, reducing muscle tension, and providing generalized warmth for comfort and relaxation. The spatial separation between heating elements enables independent thermal control, allowing different temperatures or activation states for each element.

The contoured foam cup 132 forms the outermost layer of the eye cover assembly and is secured atop the second heating element 130. As with the previously described embodiment, the foam cup 132 comprises a three-dimensionally molded or shaped structure configured to conform to the anatomical contours of the periocular region. The foam cup 132 distributes applied pressure evenly across the contact surface, enhances user comfort, protects the underlying heating elements from mechanical damage, and provides a finished aesthetic appearance.

According to one aspect, the foam cup 132 includes a fabric covering or textile layer overlying the foam material. The fabric covering provides a soft, comfortable interface against the user's skin, enhances breathability, facilitates moisture wicking, and enables easier cleaning compared to exposed foam surfaces. Suitable fabric materials include natural fibers (cotton, bamboo fiber, silk, linen), synthetic fibers (polyester, nylon, microfiber), performance fabrics with moisture-wicking properties (e.g., athletic fabrics incorporating polyester or polyamide), antimicrobial treated fabrics, or blended fiber compositions combining natural and synthetic materials.

In various embodiments, the fabric covering may be permanently attached to the foam cup 132 via adhesive bonding, thermal bonding, or stitching, or may comprise a removable cover secured via elastic bands, zipper closures, hook-and-loop fasteners, or envelope-style slip covers. Removable fabric covers enable laundering for hygiene maintenance while preserving the integrity of the underlying foam and heating elements.

In certain embodiments, the entire eye cover 104 is encased within a fabric covering or textile envelope that encloses all layers of the assembly. This complete fabric enclosure provides uniform aesthetic appearance, protects all components from contamination or moisture ingress, enables complete laundering of the exterior surface (in embodiments with removable/detachable electronics), and provides consistent tactile properties across the entire eye cover surface. The complete fabric covering may include openings or ports for electrical connections to the tethers 114a, 114b, with appropriate sealing or strain relief features at the connection points.

The fabric covering may incorporate additional functional features such as: quilting or stitched patterns providing textural interest and reinforcement; multiple fabric layers with different properties (e.g., soft inner layer against skin, moisture-resistant outer layer); thermal insulation properties directing heat toward the user rather than dissipating to the environment; or decorative elements such as colors, patterns, or embroidery enhancing aesthetic appeal.

The layers of the eye cover assembly (cushioning pad 126, first heating element 124, foam donut 128, second heating element 130, foam cup 132, and fabric covering) may be secured together using various bonding or attachment techniques including adhesive bonding (pressure-sensitive adhesive, heat-activated adhesive, or liquid adhesive), thermal welding, ultrasonic welding, radio-frequency welding, mechanical fasteners, or stitching through the layers. The bonding method is selected to provide durable attachment while maintaining flexibility of the assembly and avoiding damage to the heating elements.

While the illustrated embodiment depicts a dual heating element configuration (first heating element 124 and second heating element 130), the eye cover architecture is adaptable to various heating element quantities. In simplified embodiments, a single heating element may be employed at a selected depth within the layer stack, eliminating the complexity and cost associated with multiple heating elements while still providing effective thermal therapy. Such single-element embodiments may position the heating element at any suitable layer location, such as between the cushioning pad and foam donut, or between the foam donut and foam cup, depending on desired heat delivery characteristics.

In more complex embodiments, three or more heating elements may be incorporated at different vertical positions within the layered structure. For example, a tri-element configuration may include: a first element at the base layer for direct eyelid heating; a second element at an intermediate position for mid-depth tissue heating; and a third element at an elevated position for broad periocular heating. Additional heating elements provide increasingly fine-grained control over thermal distribution and penetration depth, enabling sophisticated thermal therapy protocols.

Furthermore, each individual heating element 124, 130 may itself comprise multiple independently controllable heating zones as previously described. For example, the first heating element 124 may include an inner zone and an outer zone, and the second heating element 130 may similarly include inner and outer zones, providing a total of four independently controllable heating zones per eye cover. This multi-element, multi-zone architecture enables highly targeted thermal therapy with precise spatial control over heat delivery.

The combination of multiple heating elements at different depths and multiple zones within each element provides a multi-dimensional heating matrix enabling sophisticated thermal delivery patterns. For instance, a user experiencing meibomian gland dysfunction might activate the inner zone of the first heating element 124 (closest to skin) to deliver concentrated heat to the eyelid margin, while leaving other zones deactivated. Conversely, a user seeking generalized warmth and relaxation might activate the outer zones of both heating elements to provide broad, gentle heating across the periocular region.

The electrical connections for the dual heating element configuration may be routed through the tethers 114a, 114b as previously described. Where multiple heating elements are employed, the electrical conductors within the tethers must accommodate the increased number of connections. For example, a dual-element configuration with two zones per element might require up to eight conductors per eye cover (or fewer if common ground architecture is employed), necessitating multi-conductor cables with appropriate insulation and strain relief.

Each heating element 124, 130 may be independently controllable via the control interface (buttons 106a, 106b), or multiple control buttons may be provided to enable separate control of each heating element. The control architecture may be configured such that: one button controls the first heating element 124 in both eye covers while another button controls the second heating element 130 in both eye covers; or separate buttons control each eye cover's heating elements independently; or buttons control heating zones across elements; or combinations thereof depending on the desired control functionality.

The heating elements 124, 130 may comprise the same or different heating technologies. For instance, the first heating element 124 might comprise a graphene heating film optimized for uniform, efficient heat generation, while the second heating element 130 might comprise a wire-based heating coil providing different thermal distribution characteristics. Alternatively, both elements may employ identical heating technology but with different resistance values, power ratings, or geometric configurations to achieve desired thermal performance.

To utilize the eye mask 100, the user positions the device such that each eye cover 104 is aligned with a corresponding eye 134. The user places the interior surface of each eye cover 104 over the periocular region surrounding each eye, with the aperture defined by the foam donut 128 aligned concentrically with the eye itself. The contoured foam cup 132 conforms to the facial anatomy, and the user adjusts the position of each eye cover 104 using the detachable fastening mechanism on the interior surface 102a of the body portion 102 to achieve optimal alignment and comfort.

Once properly positioned, the user secures the eye mask 100 in place using the lateral straps 102c, 102d extending around the head, adjusting the strap tension via the fastening mechanism to achieve comfortable but secure retention without excessive pressure. With the device securely positioned, the user activates the desired heating elements and zones using the control buttons 106a, 106b accessible on the exterior surface 102b of the body portion 102.

During use, the heating elements 124, 130 generate thermal energy that is conducted through the intervening foam layers and fabric covering to the user's periocular skin and underlying tissues. The heat promotes therapeutic effects including liquefaction of meibum within the meibomian glands, increased local blood circulation, reduction of inflammation, muscle relaxation, and symptomatic relief from dry eye, blepharitis, and related conditions. The user may wear the device for a predetermined treatment duration, typically ranging from 5 to 20 minutes, which may be controlled manually by the user or automatically by timer circuitry within the device.

After completing the thermal therapy session, the user deactivates the heating elements via the control buttons 106a, 106b, removes the eye mask 100, and stores the device until the next treatment session. If the power sources 110a, 110b are depleted, the user recharges the device using the charging interface as previously described, preparing the device for subsequent use.

In an alternative embodiment of the present disclosure, the eye mask 100 may include a steam generation system configured to provide combined heat and moisture therapy. This embodiment may be particularly beneficial for users who respond favorably to moist heat therapy or who experience enhanced comfort and tear film improvement with steam-based treatments.

In this steam-emitting embodiment, the eye mask 100 comprises: a water reservoir 136 disposed within the body portion and configured to contain a volume of water; a fill port 138 providing access to the water reservoir and steam generation assembly 136 for introducing water prior to use. The water reservoir and steam generation assembly 136 are configured to convert water into therapeutic steam during operation; and one or more vents 140 positioned to release steam toward the periocular region during use.

The water reservoir and steam generation assembly 136 is housed within the interior of the body portion and sealed to prevent leakage during normal operation. The fill port 138 allows the user to introduce water into the reservoir prior to commencing a treatment session. In certain embodiments, the fill port 138 includes a removable cap, plug, or seal element configured to close the fill port after filling, thereby preventing water leakage and maintaining internal pressure during the heating cycle.

The steam generation assembly portion of the water reservoir and steam generation assembly 136 may comprise a heating element operatively coupled to the power sources previously described. When activated, the heating element warms the water within the reservoir to generate steam. The heating element may comprise a micro-heater, vaporizing element, or resistive heating coil positioned in thermal contact with the water reservoir or immersed within the water itself.

The steam generation system of the water reservoir and steam generation assembly 136 may further include steam distribution components configured to ensure even dispersion of steam across the interior surface of the mask contacting the user's periocular region. Such distribution components may include: moisture-wicking layers that absorb and gradually release steam; porous membranes or diffusion barriers that regulate steam flow; capillary channels or microporous structures that distribute moisture evenly; or combinations thereof.

The vents 140 are strategically positioned to allow controlled release of steam toward the eyelids and periocular tissues. The vents 140 may be located near the eye covers 104, along the interior surface of the body portion, or integrated within the eye cover assemblies themselves. The size, number, and positioning of the vents are selected to maintain a humid, warm microenvironment over the eyelids while preventing excessive moisture accumulation that could cause discomfort or skin irritation.

The combination of heat and moisture provided by the steam generation system water of the reservoir and steam generation assembly 136 improves heat transfer efficiency to the meibomian glands and surrounding tissues. The moisture component hydrates the eyelid skin and softens meibomian gland secretions, potentially enhancing gland expression and therapeutic efficacy compared to dry heat alone.

In certain embodiments, the steam generation assembly operates in combination with the heating elements previously described (such as graphene heating films or resistive heating coils disposed within the eye covers). This combined configuration enables multiple operational modes selectable by the user through the control interface, including: a dry heat mode wherein only the heating elements within the eye covers 104 are activated; a moist heat mode wherein the steam generation system is activated with or without the eye cover heating elements; or a combination mode wherein both the steam generation system and eye cover heating elements operate simultaneously to provide enhanced thermal and moisture therapy.

The control interface may include additional control elements (such as buttons, switches, or settings) enabling the user to select among the available operational modes and to control steam generation independently of the heating element operation. In embodiments incorporating temperature and humidity sensing, the control circuitry may automatically regulate temperature and moisture levels based on sensor feedback to maintain safe and comfortable operating conditions.

The steam-emitting embodiment thus provides an additional therapeutic modality, offering benefits including: enhanced eyelid hydration; improved comfort for users with sensitive or dry skin; potentially improved meibomian gland warming and secretion liquefaction through combined heat and moisture application; and user-selectable therapy modes accommodating individual preferences and therapeutic requirements.

“Comprise” and variations, such as “comprising” and “comprises,” are not intended to exclude other additives, components, integers, or steps. “A”, “an,” and “the” and similar referents used herein are to be construed to cover both the singular and the plural unless their usage in context indicates otherwise. The word “exemplary” is used herein to mean “serving as an example, instance, or illustration.” Any implementation or embodiment described herein as “exemplary” is not necessarily to be construed as preferred or advantageous over other embodiments or implementations. Likewise, “embodiments” does not require that all embodiments include the discussed feature, advantage or mode of operation.

“Aspects” do not require that all aspects of the disclosure include the discussed features, advantages, or modes of operation. “Coupled” is used herein to means the direct or indirect coupling between two objects. For example, if object A physically touches or couples to object B, and object B touches or couples to object C, then objects A and C may still be considered coupled to one another, even if they do not directly physically touch each other.

One or more of the components and functions illustrated in the FIGS. may be rearranged and/or combined into a single component or embodied in several components without departing from the invention. Additional elements or components may also be added without departing from the invention. Additionally, the features described herein may be implemented in software, hardware, as a business method, and/or combination thereof.

While certain exemplary embodiments have been described and shown in the accompanying drawings, it is to be understood that such embodiments are merely illustrative of and not restrictive on the broad invention, and that this invention is not be limited to the specific constructions and arrangements shown and described, since various other modifications may occur to those ordinarily skilled in the art.

1. An eye mask apparatus, comprising:
a body portion configured to be worn over a user's eyes, the body portion having an interior surface and an exterior surface;
at least one eye cover detachably secured to the interior surface of the body portion;
at least one heating element disposed within the eye cover, the heating element comprising a plurality of independently controllable heating zones;
at least one power source secured to the body portion and electrically connected to the heating element; and
a control interface disposed on the body portion and operatively coupled to the heating element, the control interface configured to enable independent activation and deactivation of different heating zones.

2. The eye mask apparatus of claim 1, wherein each heating zone is positioned to overlay a different anatomical region of the periocular area when the eye mask is worn.

3. The eye mask apparatus of claim 1, wherein the plurality of independently controllable heating zones comprises an inner heating zone and an outer heating zone.

4. The eye mask apparatus of claim 1, wherein the control interface comprises a first control button configured to activate a first heating zone and a second control button configured to activate a second heating zone

5. The eye mask apparatus of claim 1, wherein the at least one eye cover comprises a first eye cover and a second eye cover, and wherein each control element in the control interface is configured to simultaneously control corresponding heating zones in both the first eye cover and the second eye cover.

6. The eye mask apparatus of claim 1, wherein the heating element comprises a graphene heating film, carbon fiber heating pad, or resistive heating coil.

7. The eye mask apparatus of claim 1, wherein each independently controllable heating zone is electrically isolated and separately addressable through the control interface.

8. An eye mask apparatus, comprising:
a body portion configured to be worn over a user's eyes, the body portion having an interior surface;
at least one eye cover comprising at least one heating element;
a detachable fastening mechanism configured to releasably secure the eye cover to the interior surface of the body portion and to permit repositioning of the eye cover across multiple positions on the interior surface to accommodate varying facial anatomies;
at least one power source secured to the body portion and electrically connected to the heating element; and
at least one flexible tether connecting the eye cover to the body portion, the tether providing electrical connectivity between the power source and the heating element.

9. The eye mask apparatus of claim 8, wherein the detachable fastening mechanism comprises hook-and-loop fasteners disposed on the interior surface of the body portion and on an exterior surface of the eye cover.

10. The eye mask apparatus of claim 8, wherein the at least one flexible tether comprises a first tether and a second tether connecting the eye cover to the body portion, wherein the first tether contains electrical conductors providing electrical connectivity and the second tether provides mechanical retention.

11. The eye mask apparatus of claim 8, wherein the at least one eye cover comprises:
a cushioning pad;
the heating element disposed on the cushioning pad;
an annular foam member defining an aperture and overlying the heating element; and
a contoured foam cup overlying the annular foam member.

12. The eye mask apparatus of claim 8, wherein the body portion comprises a malleable material configured to be manually deformed to conform to individual facial anatomy.

13. The eye mask apparatus of claim 8, wherein the detachable fastening mechanism permits adjustment of the eye cover position in both lateral and vertical directions relative to the body portion.

14. An eye mask apparatus, comprising:
a body portion configured to be worn over a user's eyes, the body portion having an interior surface and an exterior surface;
a first eye cover and a second eye cover, each eye cover comprising at least one heating element having a plurality of independently controllable heating zones;
a detachable fastening mechanism configured to releasably secure each eye cover to the interior surface of the body portion and to permit independent repositioning of each eye cover to accommodate varying facial anatomies;
at least one power source secured to the body portion and electrically connected to the heating elements; and
a control interface comprising at least two user-actuatable control elements, each control element configured to independently control activation of at least one heating zone in the heating elements.

15. The eye mask apparatus of claim 14, wherein the control interface is configured such that a first control element activates an inner heating zone in both the first eye cover and the second eye cover, and a second control element activates an outer heating zone in both the first eye cover and the second eye cover.

16. The eye mask apparatus of claim 14, wherein each eye cover comprises:
a cushioning pad;
a first heating element disposed on the cushioning pad;
an annular foam member overlying the first heating element; and
a second heating element disposed on the annular foam member.

17. The eye mask apparatus of claim 14, wherein the at least one power source comprises a first rechargeable battery and a second rechargeable battery, each battery independently supplying power to different heating zones or different eye covers.

18. The eye mask apparatus of claim 14, wherein the body portion further comprises:
a first lateral strap extending from a first side of the body portion; and
a second lateral strap extending from a second side of the body portion;
wherein the first and second lateral straps are configured to encircle a user's head and include a fastening mechanism for releasable attachment.

19. The eye mask apparatus of claim 14, further comprising a foam retention member overlying the at least one power source and the control interface, the foam retention member defining apertures aligned with the control elements to permit user actuation.

20. The eye mask apparatus of claim 14, further comprising:
a water reservoir disposed within the body portion;
a steam generation assembly operatively coupled to the water reservoir and to the at least one power source, the steam generation assembly configured to generate steam from water contained in the reservoir; and
at least one vent positioned to release steam toward the periocular region.
